(19) United States
(12) Patent Application Publication
(10) Pub. No.: US 2024/0315670 A1
(43) Pub. Date: Sep. 26, 2024

(54) INTRAVASCULAR ULTRASOUND CATHETER WITH BIAS CORRECTION

(71) Applicant: Boston Scientific Scimed Inc., Maple Grove, MN (US)

(72) Inventors: Paul F. Chouinard, Maple Grove, MN (US); Brady Scott Logan, Monticello, MN (US)

(73) Assignee: Boston Scientific Scimed Inc., Maple Grove, MN (US)

(21) Appl. No.: 18/612,405

(22) Filed: Mar. 21, 2024

Related U.S. Application Data
(60) Provisional application No. 63/453,869, filed on Mar. 22, 2023.

(51) Int. Cl. A61B 8/00; A61B 8/12

(52) U.S. Cl. CPC A61B 8/4477; A61B 8/12; A61B 8/445

(57) ABSTRACT

An intravascular ultrasound catheter is provided that includes multiple transducers placed facing the same radial angle or radially offset from each other. The transducers are disposed on planar surfaces that are offset from a centerline, which is orthogonal to an axis of the catheter. The planar surfaces can be offset from the centerline between −10 and 10 degrees.

### CROSS-REFERENCE TO RELATED APPLICATIONS

This application claims the benefit of U.S. Provisional Patent Application Ser. No. 63/453,869 filed Mar. 22, 2023, the disclosure of which is incorporated herein by reference.

### TECHNICAL FIELD

The present disclosure pertains to medical devices and/or medical device systems. More particularly, the present disclosure pertains to intracorporeal ultrasound catheters and transducers in such catheters.

### BACKGROUND

A wide variety of intracorporeal medical devices have been developed for medical use, for example, intravascular use. Some of these devices include guidewires, catheters, and the like. A subset of these devices includes ultrasound transducers configured to generate ultrasound signals that can be used to recreate images of the vessel. Signals from such devices (e.g., intravascular ultrasound (IVUS) devices, or the like) are routinely used to determine the diameter of vessels as well the degree and/or location of calcium or other stenosis as well as to select an appropriate intervention (e.g., stent, balloon angioplasty, etc.). However, the measurements produced by IVUS catheters presume that the catheter is parallel to the axis of the vessel. The angle of the IVUS catheter, and thus the angle of the image, is susceptible to guidewire bias which can result in a distorted image. In large diameter vessels with significant angulation, such as in the iliocaval veins of the deep pelvis, errors resulting from the bias can multiply and have a significant negative effect on the resulting image. Thus, a need exists for more accurate IVUS catheter systems which are less susceptible to error induced by guidewire bias.

### BRIEF SUMMARY

The present disclosure provides an IVUS catheter that incorporates two or more transducers at oblique angles, which can be used along with various signal processing algorithms to resolve a truer rendering of a vessel than using conventional IVUS catheters.

The disclosure can be implemented as an imaging head for an intravascular ultrasound (IVUS) catheter. The imaging head can comprise a transducer bed comprising a plurality of planar surfaces; a first ultrasound transducer disposed on a first one of the plurality of planar surfaces; and a second ultrasound transducer disposed on a second one of the plurality of planar surface.

In further examples of the imaging head, the first one of the plurality of planar surfaces and the second one of the plurality of planar surfaces are disposed on the same side of the transducer bed.

In further examples of the imaging head, the first one of the plurality of planar surfaces and the second one of the plurality of planar surfaces are radially offset from each other between 0 and 20 degrees.

In further examples of the imaging head, the first one of the plurality of planar surfaces and the second one of the plurality of planar surfaces are disposed on opposite sides of the transducer bed.

In further examples of the imaging head, the first one of the plurality of planar surfaces and the second one of the plurality of planar surfaces are radially offset from each other between 170 and 190 degrees.

In further examples of the imaging head, the first one of the plurality of planar surfaces is longitudinally offset from a centerline of the imaging head a first angle and wherein the second one of the plurality of planar surfaces is longitudinally offset from the centerline of the imaging head a second angle.

In further examples of the imaging head, the first angle is greater than or equal to −10 and less than or equal to 10 and wherein the second angle is greater than or equal to −10 and less than 10 degrees.

In further examples of the imaging head, the first angle and the second angle are the same.

In further examples of the imaging head, the first angle is greater than or equal to −10 and less than or equal to 0 and wherein the second angle is greater than or equal to 0 and less than or equal to 10.

In further examples, the imaging head, can comprise a third ultrasound transducer disposed on a third one of the plurality of planar surface.

In further examples of the imaging head, the third one of the plurality of planar surfaces is disposed on an opposite side of the transducer bed from at least the first one of the plurality of planar surfaces.

In further examples, the imaging head can comprise a fourth ultrasound transducer disposed on a fourth one of the plurality of planar surfaces.

In further examples of the imaging head, the third one of the plurality of planar surfaces and the fourth one of the plurality of planar surfaces are disposed on an opposite side of the transducer bed from the first one of the plurality of planar surfaces and the second one of the plurality of planar surfaces.

In further examples, the imaging head can comprise an ultrasound receiver disposed on a third one of the plurality of planar surface.

In further examples of the imaging head, the first one of the plurality of planar surfaces and the second one of the plurality of planar surfaces are disposed on the same side of the transducer bed, and wherein the third one of the plurality of planar surfaces is disposed on an opposite side of the transducer bed from the first one of the plurality of planar surfaces and the second.

The disclosure can be implemented as an imaging head for an intravascular ultrasound (IVUS) catheter. The imaging head can comprise a transducer bed comprising a plurality of planar surfaces; a first ultrasound transducer disposed on a first one of the plurality of planar surfaces; a second ultrasound transducer disposed on a second one of the plurality of planar surface; and a third ultrasound transducer disposed on a third one of the plurality of planar surface.

In further examples of the imaging head, the third one of the plurality of planar surfaces is disposed on an opposite side of the transducer bed from at least the first one of the plurality of planar surfaces.

In further examples, the imaging head can comprise a fourth ultrasound transducer disposed on a fourth one of the plurality of planar surfaces.

In further examples of the imaging head, the third one of the plurality of planar surfaces and the fourth one of the plurality of planar surfaces are disposed on an opposite side of the transducer bed from the first one of the plurality of planar surfaces and the second one of the plurality of planar surfaces.

In further examples, the imaging head can comprise an ultrasound receiver disposed on a third one of the plurality of planar surface.

In further examples of the imaging head, the first one of the plurality of planar surfaces and the second one of the plurality of planar surfaces are disposed on the same side of the transducer bed, and wherein the third one of the plurality of planar surfaces is disposed on an opposite side of the transducer bed from the first one of the plurality of planar surfaces and the second.

The disclosure can be implemented as an intravascular ultrasound (IVUS) catheter. The IVUS catheter can comprise a catheter sheath; a drive cable configured to be coupled to a motor drive unit; and an imaging core coupled to the drive cable, the imaging core comprising: a transducer bed comprising a plurality of planar surfaces; a first ultrasound transducer disposed on a first one of the plurality of planar surfaces; and a second ultrasound transducer disposed on a second one of the plurality of planar surface.

In further examples of the IVUS catheter, the first one of the plurality of planar surfaces and the second one of the plurality of planar surfaces are disposed on the same side of the transducer bed.

In further examples of the IVUS catheter, the first one of the plurality of planar surfaces and the second one of the plurality of planar surfaces are radially offset from each other between 0 and 20 degrees.

In further examples of the IVUS catheter, the first one of the plurality of planar surfaces and the second one of the plurality of planar surfaces are disposed on opposite sides of the transducer bed.

In further examples of the IVUS catheter, the first one of the plurality of planar surfaces and the second one of the plurality of planar surfaces are radially offset from each other between 170 and 190 degrees.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates an embodiment of an intravascular imaging system.

FIG. 2A illustrates an embodiment of portions of the intravascular imaging system of FIG. 1 in greater detail.

FIG. 2B illustrates an embodiment of another portion of the intravascular imaging system of FIG. 1 in greater detail.

FIG. 3 illustrates a distal end of a conventional IVUS catheter.

FIG. 4A shows an illustrative iliocaval vein.

FIG. 4B illustrates guidewire bias in the iliocaval vein of FIG. 4A.

FIG. 4C illustrates the actual lumen boundary of the iliocaval vein of FIG. 4A along with a rendering of the lumen boundary based on IVUS in a first region.

FIG. 4D illustrates the actual lumen boundary of the iliocaval vein of FIG. 4A along with a rendering of the lumen boundary based on IVUS in a second region.

FIG. 5 illustrates a distal end of an IVUS catheter according to at least one embodiment.

FIG. 6 illustrates a distal end of an IVUS catheter according to at least one embodiment.

FIG. 7 illustrates a distal end of yet another IVUS catheter according to at least one embodiment.

FIG. 8 illustrates a distal end of still another IVUS catheter according to at least one embodiment.

FIG. 9 illustrates a distal end of yet a different IVUS catheter according to at least one embodiment.

FIG. 10 illustrates a distal end of another different IVUS catheter according to at least one embodiment.

FIG. 11 illustrates graph showing error resulting from catheter-vessel obliquity for a conventional IVUS catheter contrasted with an IVUS catheter according to the present disclosure.

### DETAILED DESCRIPTION

As noted, numerous imaging modalities exist to assess vascular lesions, for example, magnetic resonance imaging (MRI), computerized tomography (CT), intra vascular ultrasound (IVUS), optical coherence tomography (OCT), optical coherence elastography (OCE) and spectroscopy can give insight to the degree that a vascular lesion varies from healthy tissue. The present disclosure is directed to IVUS signal acquisition and particularly to transducer placement in an IVUS catheter. However, prior to discussing such embodiments, an overall IVUS system is described.

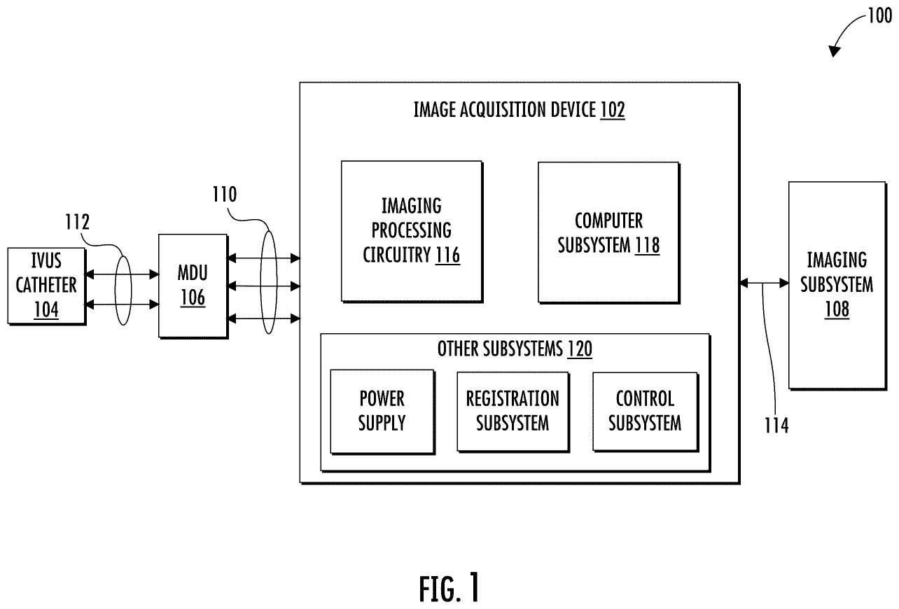

FIG. 1 illustrates an example IVUS imaging system 100. The IVUS imaging system 100 includes an image acquisition device 102, an IVUS catheter 104, a motor drive unit (MDU) 106, and an imaging subsystem 108. The image acquisition device 102 is coupled to the IVUS catheter 104 via the MDU 106 and is also coupled to the 108. In particular, the image acquisition device 102 is coupled to the MDU 106 via the MDU bus 110 while the MDU 106 is coupled to the IVUS catheter 104 via the catheter bus 112. In some embodiments, the MDU bus 110 and the catheter bus 112 can be transmission lines (or other conductors) arranged to convey signals between the various components. For example, the MDU bus 110 and catheter bus 112 can be arranged to transmit radio frequency signals (e.g., control signals, ultrasound pulse generation signals, ultrasound signals, or the like) between the indicated components of the IVUS imaging system 100.

In general, the image acquisition device 102 is configured to control the MDU 106 and receive signals from the IVUS catheter 104, via the MDU 106. Further, the image acquisition device 102 is configured to process the received signals to generate images and convey the images to the imaging subsystem 108. To that end, the image acquisition device 102 is coupled to the imaging subsystem 108 via the imaging subsystem bus 114, which can be a wired connection or a wireless connection. As a specific example, imaging subsystem bus 114 can be an Ethernet connection. In some examples, the imaging subsystem 108 can be a display, a tablet computer, or other device configured to display images rendered by image acquisition device 102. It is noted that although the imaging subsystem 108 is depicted external to image acquisition device 102, with some embodiments, imaging subsystem 108 can be incorporated into the same housing as image acquisition device 102.

The image acquisition device 102 includes an imaging processing circuitry 116, computer subsystem 118, and other subsystems 120. As indicated above, the present disclosure provides an improved IVUS catheter and transducer arrangement, which can be implemented as part of the image acquisition device 102, and particularly as part of the IVUS catheter 104. However, a general description of the components of the IVUS imaging system 100 and the image acquisition device 102 is provided prior to detailing the transducer arrangements with which the present disclosure is directed.

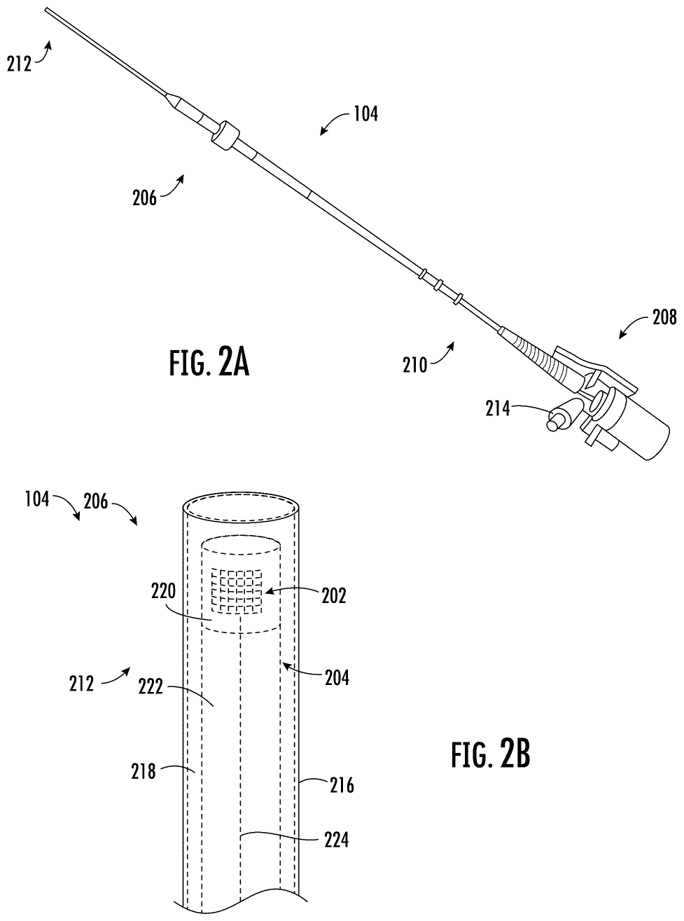

FIG. 2A and FIG. 2B illustrate a side and perspective view of the IVUS catheter 104 of the IVUS imaging system 100 of FIG. 1. The other subsystems 120 is configured to power MDU 106 and send signal to IVUS catheter 104 and particularly one or more transducers 202 disposed in the IVUS catheter 104 to cause the IVUS catheter 104 to emit ultrasound signals.

Further, mechanical energy from MDU 106 may be used to drive an imaging core 204 disposed in the IVUS catheter 104. The one or more transducers 202 are further configured to receive reflected signals (e.g., echo signals, or the like) responsive to emitting ultrasound signals. These reflected signals are transmitted to image acquisition device 102 via catheter bus 112, the MDU 106, and MDU bus 110 for processing by imaging processing circuitry 116 and computer subsystem 118.

In some embodiments, other subsystems 120 can be configured to control at least one of the frequency or duration of the electrical pulses transmitted from image acquisition device 102 to MDU 106 to control, for example, the rotation rate of the imaging core 204 by the MDU 106 or the velocity or length of the pullback of the imaging core 204 by the MDU 106.

The IVUS catheter 104 includes an elongated member 206 and a hub 208. The elongated member 206 includes a proximal end 210 and a distal end 212. The proximal end 210 of the elongated member 206 can be coupled to the hub 208 and the distal end 212 of the elongated member 206 is configured and arranged for percutaneous insertion into a patient. Optionally, the IVUS catheter 104 may define at least one flush port, such as flush port 214. The flush port 214 may be defined in the hub 208. The hub 208 may be configured and arranged to couple to the MDU 106 of IVUS imaging system 100.

In some instances, the elongated member 206 and the hub 208 are formed as a unitary body. In other instances, the elongated member 206 and the catheter hub 208 are formed separately and subsequently assembled.

FIG. 2B is a perspective view of one embodiment of the distal end 212 of the elongated member 206 of the IVUS catheter 104. The elongated member 206 includes a sheath 216 with a longitudinal axis (e.g., a central longitudinal axis extending axially through the center of the sheath 216 and/or the IVUS catheter 104) and a lumen 222. An imaging core 224 is disposed in the lumen 218. The imaging core 204 includes an imaging device 220 coupled to a distal end of a driveshaft 222 that is rotatable either manually or using a computer-controlled drive mechanism (e.g., MDU 106). The one or more transducers 202 may be mounted to the imaging device 220 and employed to transmit and receive acoustic signals. The sheath 216 may be formed from any flexible, biocompatible material suitable for insertion into a patient. Examples of suitable materials include, for example, polyethylene, polyurethane, plastic, spiral-cut stainless steel, nitinol hypotube, and the like or combinations thereof.

In some embodiments, for example as shown in these figures, an array of transducers 202 are mounted to the imaging device 220. Alternatively, a single transducer may be employed. Any suitable number of transducers 202 can be used. For example, there can be two, three, four, five, six, seven, eight, nine, ten, twelve, fifteen, sixteen, twenty, twenty-five, fifty, one hundred, five hundred, one thousand, or more transducers. As will be recognized, other numbers of transducers may also be used. When a plurality of transducers 202 are employed, the transducers 202 can be configured into any suitable arrangement including, for example, an annular arrangement, a rectangular arrangement, or the like.

The one or more transducers 202 may be formed from materials capable of transforming applied electrical pulses to pressure distortions on the surface of the one or more transducers 230, and vice versa. Examples of suitable materials include piezoelectric ceramic materials, piezocomposite materials, piezoelectric plastics, barium titanates, lead zirconate titanates, lead metaniobates, polyvinylidene fluorides, and the like. Other transducer technologies include composite materials, single-crystal composites, and semiconductor devices (e.g., capacitive micromachined ultrasound transducers (“cMUT”), piezoelectric micromachined ultrasound transducers (“pMUT”), or the like).

The pressure distortions on the surface of the one or more transducers 202 form acoustic pulses of a frequency based on the resonant frequencies of the one or more transducers 202. The resonant frequencies of the one or more transducers 202 may be affected by the size, shape, and material used to form the one or more transducers 202. The one or more transducers 202 may be formed in any shape suitable for positioning within the IVUS catheter 104 and for propagating acoustic pulses of a desired frequency in one or more selected directions. For example, transducers may be disc-shaped, block-shaped, rectangular-shaped, oval-shaped, and the like. The one or more transducers may be formed in the desired shape by any process including, for example, dicing, dice and fill, machining, microfabrication, and the like.

As an example, each of the one or more transducers 202 may include a layer of piezoelectric material sandwiched between a matching layer and a conductive backing material formed from an acoustically absorbent material (e.g., an epoxy substrate with tungsten particles). During operation, the piezoelectric layer may be electrically excited to cause the emission of acoustic pulses.

The one or more transducers 202 can be used to form a radial cross-sectional image of a surrounding space. Thus, for example, when the one or more transducers 202 are disposed in the IVUS catheter 104 and inserted into a blood vessel of a patient, the one or more transducers 202 may be used to capture acoustic signals to be processed by image acquisition device 102, and particularly by the analog front end (AFE) described herein.

The imaging core 204 is rotated about the longitudinal axis of the IVUS catheter 104. As the imaging core 204 rotates, the one or more transducers 202 emit acoustic signals in different radial directions (e.g., along different radial scan lines). For example, the one or more transducers 202 can emit acoustic signals at regular (or irregular) increments, such as 256 radial scan lines per revolution, or the like. It will be understood that other numbers of radial scan lines can be emitted per revolution, instead.

When an emitted acoustic pulse with sufficient energy encounters one or more medium boundaries, such as one or more tissue boundaries, a portion of the emitted acoustic pulse is reflected to the emitting transducer as an echo pulse. Each echo pulse that reaches a transducer with sufficient energy to be detected is transformed to an electrical signal in the receiving transducer. The one or more transformed electrical signals are transmitted to the imaging processing circuitry 116 of image acquisition device 102 where it is processed and digitized. The digitized signals can be communicated to computer subsystem 118 and used to form images of the vessel, which images can be displayed on imaging subsystem 108. In some instances, the rotation of the imaging core 204 is driven by the MDU 106, which itself is controlled by other subsystems 120.

When the one or more transducers 202 are rotated about the longitudinal axis (or parallel to the longitudinal axis) of the distal end of the IVUS catheter 104 emitting acoustic pulses, a plurality of images can be formed that collectively form a radial cross-sectional image (e.g., a tomographic image) of a portion of the region surrounding the one or more transducers 202, such as the walls of a blood vessel of interest and tissue surrounding the blood vessel. The imaging core 204 may also move longitudinally along the blood vessel within which the IVUS catheter 104 is inserted so that a plurality of cross-sectional images may be formed along a longitudinal length of the blood vessel. During an imaging procedure the one or more transducers 202 may be retracted (e.g., pulled back) along the longitudinal length of the IVUS catheter 104. The IVUS catheter 104 can include at least one telescoping section that can be retracted during pullback of the one or more transducers 202. In some instances, the MDU 106 drives the pullback of the imaging core 204 within the IVUS catheter 104. The MDU 106 pullback distance of the imaging core 204 can be any suitable distance including, for example, at least 5 cm, 10 cm, 15 cm, 20 cm, 25 cm, or more. The entire IVUS catheter 104 can be retracted during an imaging procedure either with or without the imaging core 204 moving longitudinally independently of the IVUS catheter 104.

The quality of an image produced at different depths from the one or more transducers 202 may be affected by one or more factors including, for example, bandwidth, transducer focus, beam pattern, as well as the frequency of the acoustic pulse. The frequency of the acoustic pulse output from the one or more transducers 202 may also affect the penetration depth of the acoustic pulse output from the one or more transducers 202. In general, as the frequency of an acoustic pulse is lowered, the depth of the penetration of the acoustic pulse within patient tissue increases. In some instances, the intravascular treatment IVUS imaging system 100 operates within a frequency range of 5 MHz to 200 MHZ.

One or more conductors 224 can electrically couple the transducers 202 and catheter bus 112. In such a manner, the electrical signals captured by the transducer 202 can be received by the imaging processing circuitry 116 of the image acquisition device 102.

It is noted that the IVUS imaging system 100 described above uses mechanical rotation of the transducer 202 (e.g., via the MDU 106). However, other IVUS imaging systems may include arrays of transducers, where the transducers are positioned perpendicular to the axis of the catheter positioned such that rotation of the transducers is not necessary. In such a case, the IVUS imaging system would not need an MDU. As introduced above, the present disclosure provides several exemplary transducer placements, which mitigate effects of guidewire bias. Although the examples are described with reference to the IVUS imaging systems 100 and a rotating transducer setup, the exemplary placement described in this disclosure can be applied to fixed array IVUS catheters and corresponding imaging systems.

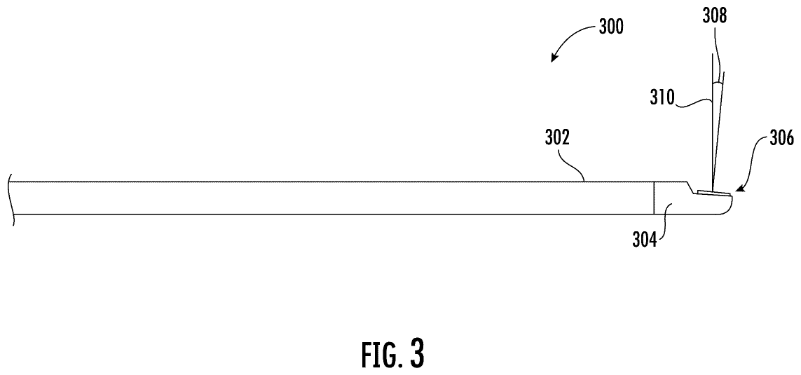

FIG. 3 illustrates an example of a distal end of a conventional IVUS catheter 300. IVUS catheter 300 includes an imaging core 302, an imaging head 304, and a transducer 306 set in the imaging head 304 at an angle 308. As can be seen, the angle 308 is offset from the vertical or radial angle of the imaging head 304. Conventionally, this angle is 5 degrees. As introduced above, conventional IVUS catheters (e.g., IVUS catheter 300) are susceptible to guidewire bias.

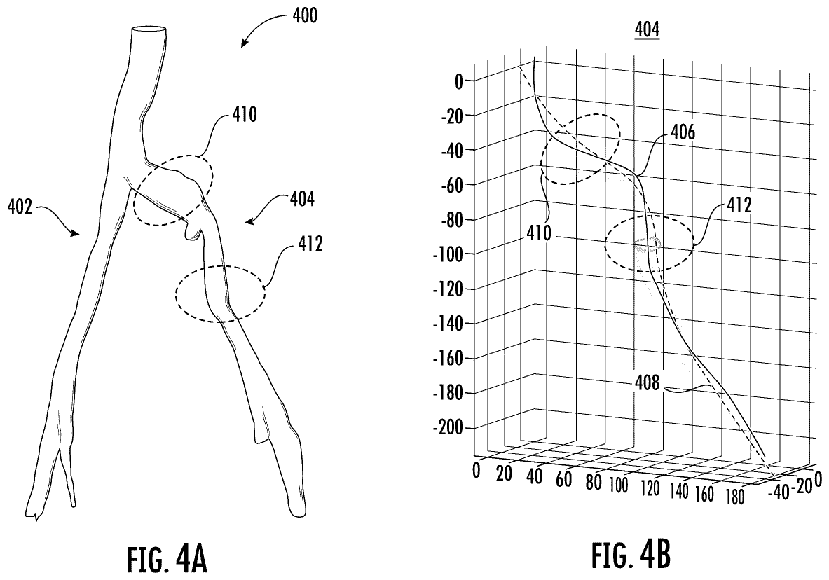

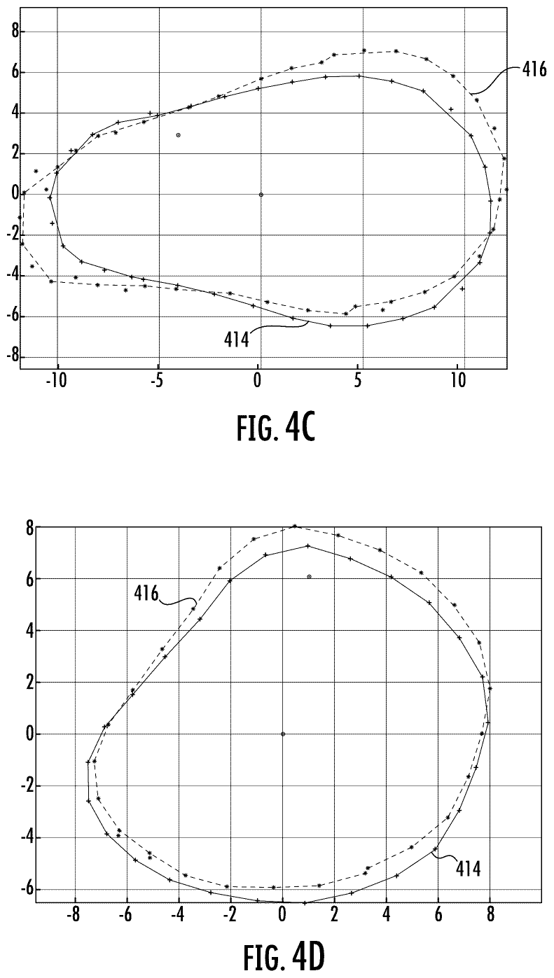

FIG. 4A illustrates an example iliocaval vein 400 having a right iliac vein 402 and a left iliac vein 404. The iliocaval vein 400 is in the pelvis region and can be subject to several maladies with which intracorporeal imaging (e.g., IVUS imaging) may be helpful to diagnose and/or treat. However, due to the size and shape of the iliocaval vein 400, images may become distorted due to what is referred to as guidewire bias.

FIG. 4B illustrates the left iliac vein 404 with a guidewire disposed in the left iliac vein 404. The true centerline 406 of the left iliac vein 404 is highlighted along with the guidewire path 408. As can be seen, due to the anatomy of the left iliac vein 404, the true centerline 406 and guidewire path 408 diverge at various points along the left iliac vein 404. This divergence is what leads to guidewire bias. For example, where the guidewire path 408 diverges from true centerline 406, the distance from the transducer of an IVUS catheter coupled to the guidewire to the wall of the left iliac vein 404 will differ from one side to the other. Further, the angle of incidence of the ultrasound signal on the wall of the left iliac vein 404 will differ from one side of the left iliac vein 404 to the other where the guidewire path 408 diverges from the true centerline 406. The greater the divergence, the greater the difference in distance and angle of incidence between transducer and walls on either side of left iliac vein 404. This difference in distance and angle of incidence leads to distortions in the resultant ultrasound images.

For example, regions 410 and 412 are highlighted on left iliac vein 404. FIG. 4C shows an image of the actual lumen boundary 414 and IVUS lumen boundary 416 from region 410. As can be seen, the guidewire path 408 diverges significantly from true centerline 406 in region 410. As a result, the ultrasound image from region 410 will be distorted. This is manifest in the lumen boundaries. IVUS lumen boundary 416 is significantly larger than actual lumen boundary 414 in region 410.

Conversely, FIG. 4D illustrates image of the actual lumen boundary 414 and IVUS lumen boundary 416 from region 412. As can be seen, the guidewire path 408 does not diverge as much from true centerline 406 in region 412 as compared to region 410. As a result, the ultrasound image from region 412 will be significantly less distorted. This is manifest in the lumen boundaries. IVUS lumen boundary 416 more closely matches the actual lumen boundary 414 in region 412.

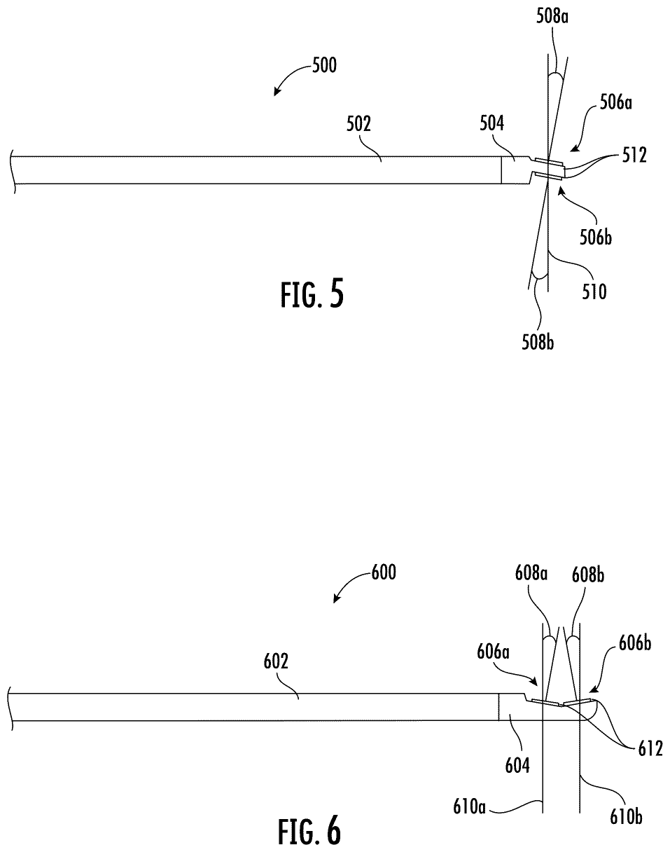

FIG. 5 illustrates an example of a distal end of an IVUS catheter 500, arranged according to at least one embodiment of the present disclosure. IVUS catheter 500 includes an imaging core 502, an imaging head 504, and transducers 506a and 506b set in the imaging head 504 at angles 508a and 508b, offset from the centerline 510.

As can be seen, IVUS catheter 500 includes multiple transducers (e.g., transducers 506a and 506b) radially offset from each other. For example, as can be seen, transducers 506a and 506b are radially offset from each other approximately 180 degrees. Further, the transducers 506a and 506b are placed at angles offset from the centerline 510. As used herein, the term “centerline” means a line perpendicular to the longitudinal axis of the IVUS catheter 500. As such, when the IVUS catheter 500 is non-parallel to the center axis of the vessel (e.g., left iliac vein 404, or the like) one transducer (e.g., transducer 506a, or the like) will be biased more perpendicularly to the wall of the vessel than the other (e.g., transducer 506b, or the like). The transducer that is more perpendicular to the vessel wall produces a higher-contrast image and shows the vein wall closer to the catheter. In contrast, the transducer that is more oblique to the vessel wall produces a lower-contrast image with the vein wall further from the catheter.

The imaging head 504 includes a shaped transducer bed comprising several planar surfaces 512 formed at angles to the centerline 510, which provide a platform and support for the transducers 506a and 506b. With some examples, the angles 508a and 508b are between −10 and 10 degrees. Further, the angles 508a and 508b need not be the same. For example, angle 508a can be 5 degrees while angle 508b can be 10 degrees. Although beyond the scope of this disclosure, image processing software can render the composite vessel image using algorithms that will use both signals (e.g., from transducers 506a and 506b).

Further, it is noted that the planar surfaces 512 can be on the same side of the imaging head 504 (e.g., see FIG. 6, or the like) or on opposite sides of the imaging head 504 (e.g., as shown in FIG. 5). Further, with some embodiments, the planar surface 512 can be radially offset from each other. For example, one of planar surface 512 can be radially offset between 170 and 190 degrees from the other planar surface 512.

FIG. 6 illustrates an example of a distal end of an IVUS catheter 600, arranged according to at least one embodiment of the present disclosure. IVUS catheter 600 includes an imaging core 602, an imaging head 604, and transducers 606a and 606b set in the imaging head 604 at angles 608a and 608b, offset from the centerlines 610a and 610b, respectively. As can be seen, IVUS catheter 600 includes multiple transducers, which are offset from a viewing plane of the imaging head 604, where the viewing plane is a plane perpendicular to the longitudinal axis of the imaging head 604. In this case, one transducer is offset a positive angle from the viewing plane while the other is offset a negative angle from the viewing plane. In this example, the transducers 606a and 606b angled towards each other. With some examples, the angles 608a and 608b are between −10 and 10 degrees. Further, the angles 608a and 608b need not be the same.

As noted above, with some examples, the planar surface 612 on which the transducers 606a and 606b are disposed is not radially offset. In other examples, the planar surface 612 are slightly radially offset, for example, between 0 and 20 degrees from each other.

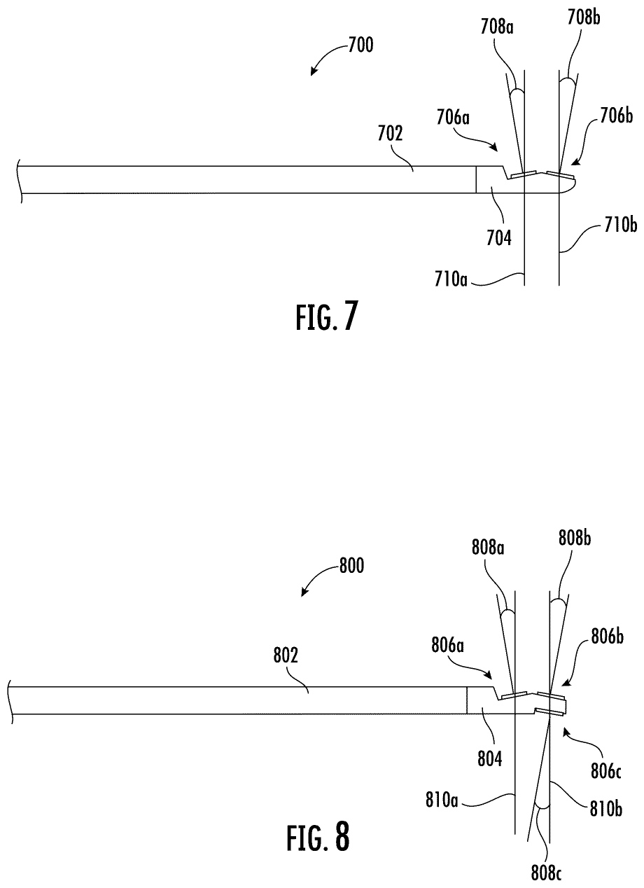

FIG. 7 illustrates an example of a distal end of an IVUS catheter 700, arranged according to at least one embodiment of the present disclosure. IVUS catheter 700 includes an imaging core 702, an imaging head 704, and transducers 706a and 706b set in the imaging head 704 at angles 708a and 708b, offset from the centerlines 710a and 710b, respectively. As can be seen, IVUS catheter 700 includes multiple transducers, offset from each other in the viewing plane. In this example, the transducers 706a and 706b angled away from each other. With some examples, the angles 708a and 708b are between −10 and 10 degrees. Further, the angles 708a and 708b need not be the same.

FIG. 8 illustrates an example of a distal end of an IVUS catheter 800, arranged according to at least one embodiment of the present disclosure. IVUS catheter 800 includes an imaging core 802, an imaging head 804, and transducers 806a, 806b, and 806c set in the imaging head 804. As can be seen, IVUS catheter 800 includes multiple transducers, radially offset from each other. IVUS catheter 800 includes transducers 806a and 806b facing substantially the same radial angle and further offset from the centerlines 810a and 810b, respectively. Further, IVUS catheter 800 includes transducer 806c radially offset from transducers 806a and 806b (e.g., approximately 180 degrees, or the like) and placed at an angle 808c offset from the centerline 810b. It is noted that although transducers 806a and 806b are depicted facing away from each other, they could be positioned facing each other (e.g., like transducers 606a and 606b of FIG. 6). With some examples, the angles 808a, 808b, and 808c are between −10 and 10 degrees. Further, the angles 808a, 808b, and 808c need not be the same.

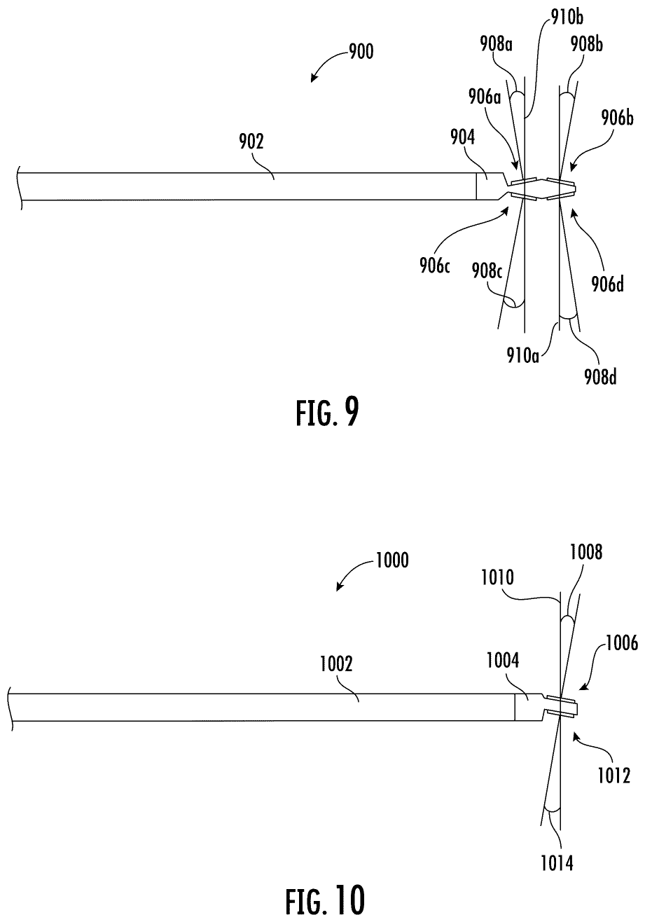

FIG. 9 illustrates an example of a distal end of an IVUS catheter 900, arranged according to at least one embodiment of the present disclosure. IVUS catheter 900 includes an imaging core 902, an imaging head 904, and transducers 906a, 906b, 906c, and 906d set in the imaging head 904. As can be seen, IVUS catheter 900 includes multiple transducers, radially offset from each other. IVUS catheter 900 includes transducers 906a and 906b placed at approximately the same angle radially and offset from the centerlines 910a and 910b, respectively. Further, IVUS catheter 900 includes transducers 906c and 906d radially offset from transducers 906a and 906b (e.g., approximately 180 degrees, or the like) and placed at angle angles 908c and 908d, offset from the centerlines 910a and 910b, respectively. It is noted that although these transducers are depicted facing away from each other, they could be positioned facing each other (e.g., like transducers 606a and 606b of FIG. 6). With some examples, the angles 908a, 908b, 908c, and 908d are between −10 and 10 degrees. Further, the angles 908a, 908b, 908c, and 908d need not be the same.

FIG. 10 illustrates an example of a distal end of an IVUS catheter 1000, arranged according to at least one embodiment of the present disclosure. IVUS catheter 1000 includes an imaging core 1002, an imaging head 1004 and a transducer 1006 set in the imaging head 1004 at angle 1008 offset from centerline 1010. Further, IVUS catheter 1000 includes an ultrasound receiver 1012 set in the imaging head on the opposite side from the transducer 1006 and set at angle 1014 offset from the centerline 1010. With some examples, the angles 1008 and 1014 are between −10 and 10 degrees. Further, the angles 1008 and 1014 need not be the same.

It is noted that other embodiments described herein can have one or more transducers and two or more receivers. As another example, the devices herein can have two or more transducers and one or more receivers. For example, transducer 806a and/or transducer 806c of IVUS catheter 800 of FIG. 8 could be an ultrasound receiver.

As discussed above, the conventional IVUS catheters with a single transducer set in the imaging head suffer from guidewire bias. More specifically, the catheter-vessel obliquity, or the deviation of the vessel centerline (e.g., true centerline 406) from the catheter centerline (e.g., guidewire path 408) can range from 0 to 45 degrees. The catheter-vessel obliquity may have a mean of 10 to 15 degrees and can be significantly higher in areas of high change in curvature and confluences. This leads to a high degree of guidewire bias as explained above.

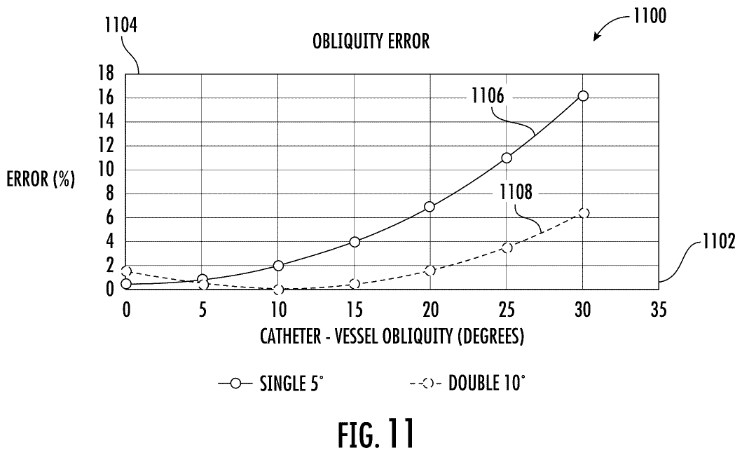

High bias in regions where sizing is critical can lead to incorrect measurements (e.g., balloon or stent selection). However, the present disclosure provides a significant reduction in obliquity error. For example, FIG. 11 illustrates a graph 1100 showing catheter-vessel obliquity in degrees on the x axis 1102 and error in percent on the y axis 1104 for two different IVUS catheters. Plot 1106 shows the error against catheter-vessel obliquity for a conventional IVUS catheter having a single transducer placed at 5 degrees offset from the centerline (e.g., IVUS catheter 300, or the like). Conversely, plot 1108 shows the error against catheter-vessel obliquity for an IVUS catheter according to the present disclosure, particularly, one that has two transducer placed at 10 degrees offset from the centerline (e.g., IVUS catheter 500, or the like).

As can be seen, the error for the IVUS catheter according to the present disclosure is significantly less as the catheter-vessel obliquity increases.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An imaging head for an intravascular ultrasound (IVUS) catheter, comprising:
a transducer bed comprising a plurality of planar surfaces;
a first ultrasound transducer disposed on a first one of the plurality of planar surfaces; and
a second ultrasound transducer disposed on a second one of the plurality of planar surfaces.

2. The imaging head of claim 1, wherein the first one of the plurality of planar surfaces and the second one of the plurality of planar surfaces are disposed on the same side of the transducer bed.

3. The imaging head of claim 2, wherein the first one of the plurality of planar surfaces and the second one of the plurality of planar surfaces are offset from each other between 0 and 20 degrees in a viewing plane, wherein the viewing is perpendicular to a longitudinal axis of the transducer bed.

4. The imaging head of claim 1, wherein the first one of the plurality of planar surfaces and the second one of the plurality of planar surfaces are disposed on opposite sides of the transducer bed.

5. The imaging head of claim 4, wherein the first one of the plurality of planar surfaces and the second one of the plurality of planar surfaces are radially offset from each other between 170 and 190 degrees.

6. The imaging head of claim 1, wherein the first one of the plurality of planar surfaces is longitudinally offset from a centerline of the imaging head a first angle and wherein the second one of the plurality of planar surfaces is longitudinally offset from the centerline of the imaging head a second angle.

7. The imaging head of claim 6, wherein the first angle is greater than or equal to −10 and less than or equal to 10 and wherein the second angle is greater than or equal to −10 and less than 10 degrees.

8. The imaging head of claim 7, wherein the first angle is greater than or equal to −10 and less than or equal to 0 and wherein the second angle is greater than or equal to 0 and less than or equal to 10.

9. The imaging head of claim 8, wherein the first angle and the second angle are the same.

10. An imaging head for an intravascular ultrasound (IVUS) catheter, comprising:
a transducer bed comprising a plurality of planar surfaces;
a first ultrasound transducer disposed on a first one of the plurality of planar surfaces;
a second ultrasound transducer disposed on a second one of the plurality of planar surfaces; and
a third ultrasound transducer disposed on a third one of the plurality of planar surface.

11. The imaging head of claim 10, wherein the third one of the plurality of planar surfaces is disposed on an opposite side of the transducer bed from at least the first one of the plurality of planar surfaces.

12. The imaging head of claim 10, comprising a fourth ultrasound transducer disposed on a fourth one of the plurality of planar surfaces.

13. The imaging head of claim 12, wherein the third one of the plurality of planar surfaces and the fourth one of the plurality of planar surfaces are disposed on an opposite side of the transducer bed from the first one of the plurality of planar surfaces and the second one of the plurality of planar surfaces.

14. The imaging head of claim 10, comprising an ultrasound receiver disposed on a third one of the plurality of planar surfaces.

15. The imaging head of claim 14, wherein the first one of the plurality of planar surfaces and the second one of the plurality of planar surfaces are disposed on the same side of the transducer bed, and wherein the third one of the plurality of planar surfaces is disposed on an opposite side of the transducer bed from the first one of the plurality of planar surfaces and the second.

16. An intravascular ultrasound (IVUS) catheter, comprising:
a catheter sheath;
a drive cable configured to be coupled to a motor drive unit; and
an imaging core coupled to the drive cable, the imaging core comprising:
a transducer bed comprising a plurality of planar surfaces;
a first ultrasound transducer disposed on a first one of the plurality of planar surfaces; and
a second ultrasound transducer disposed on a second one of the plurality of planar surfaces.

17. The IVUS catheter of claim 16, wherein the first one of the plurality of planar surfaces and the second one of the plurality of planar surfaces are disposed on the same side of the transducer bed.

18. The IVUS catheter of claim 17, wherein the first one of the plurality of planar surfaces and the second one of the plurality of planar surfaces are offset from each other between 0 and 20 degrees in a viewing plane, wherein the viewing is perpendicular to a longitudinal axis of the transducer bed.

19. The IVUS catheter of claim 16, wherein the first one of the plurality of planar surfaces and the second one of the plurality of planar surfaces are disposed on opposite sides of the transducer bed.

20. The IVUS catheter of claim 19, wherein the first one of the plurality of planar surfaces and the second one of the plurality of planar surfaces are radially offset from each other between 170 and 190 degrees.
